(19) United States
(12) Patent Application Publication
(10) Pub. No.: US 2024/0264102 A1
(43) Pub. Date: Aug. 8, 2024

(54) GAS SENSOR

(71) Applicant: TDK Corporation, Tokyo (JP)

(72) Inventors: Sadaharu YONEDA, Tokyo (JP); Yutaka MATSUO, Tokyo (JP); Yoshio KAITA, Tokyo (JP)

(21) Appl. No.: 18/433,793

(22) Filed: Feb. 6, 2024

(30) Foreign Application Priority Data
Feb. 8, 2023 (JP) 2023-017291
Nov. 7, 2023 (JP) 2023-189887

(51) Int. Cl. G01N 25/18; G01N 33/00

(52) U.S. Cl. CPC G01N 25/18; G01N 33/0027

(57) ABSTRACT

Disclosed herein is a gas sensor that includes: a gas sensor part including a thermosensitive element, the gas sensor part being configured to generate a gas detection signal according to a concentration of a gas to be detected; a temperature sensor part configured to generate a temperature detection signal according to environmental temperature; a heater configured to heat the thermosensitive element; and a signal processing circuit configured to: control a heating temperature of the heater according to the temperature detection signal obtained at a first timing; and calculate the concentration of the gas to be detected according to the temperature detection signal obtained at a second timing that is later than the first timing and the gas detection signal obtained at a third timing at which the thermosensitive element is heated by the heater.

### CROSS-REFERENCE TO RELATED APPLICATION

This application claims the benefit of Japanese Patent Application No. 2023-017291, filed on Feb. 8, 2023, and Japanese Patent Application No. 2023-189887, filed on Nov. 7, 2023, the entire disclosure of which are incorporated by reference herein.

### BACKGROUND OF THE ART

### Field of the Art

The present disclosure relates to a gas sensor and, more particularly, to a gas sensor provided with a thermosensitive element such as a thermistor and a heater for heating the thermosensitive element.

### Description of Related Art

Japanese Patent No. 7,070,175 discloses a gas sensor provided with a thermistor and a heater for heating the thermistor. The gas sensor disclosed in Japanese Patent No. 7,070,175 controls heating temperature of the heater according to environmental temperature such that the thermistor is heated to a desired temperature.

However, an environmental temperature change has influence not only on the heater's heating temperature but also on the reference level of a gas detection signal corresponding to the concentration of a gas to be detected.

### SUMMARY

The present disclosure describes a technology for allowing a gas sensor provided with a thermosensitive element such as a thermistor and a heater for heating the thermosensitive element to accurately detect a gas concentration even with a change in environmental temperature.

A gas sensor according to one embodiment of the present disclosure includes: a gas sensor part including a thermosensitive element, the gas sensor part being configured to generate a gas detection signal according to a concentration of a gas to be detected; a temperature sensor part configured to generate a temperature detection signal according to environmental temperature; a heater configured to heat the thermosensitive element; and a signal processing circuit configured to: control a heating temperature of the heater according to the temperature detection signal obtained at a first timing; and calculate the concentration of the gas to be detected according to the temperature detection signal obtained at a second timing that is later than the first timing and the gas detection signal obtained at a third timing at which the thermosensitive element is heated by the heater.

### BRIEF DESCRIPTION OF THE DRAWINGS

The above features and advantages of the present disclosure will be more apparent from the following description of certain preferred embodiments taken in conjunction with the accompanying drawings, in which:

FIG. 1 is a circuit diagram illustrating the configuration of a gas sensor 1 according to a first embodiment of the technology according to the present disclosure;

FIG. 2 is a flowchart for explaining the operation of the gas sensor 1;

FIG. 3 is a timing chart for explaining the operation of the gas sensor 1;

FIG. 4 is a timing chart for explaining influence of a temperature error when the environmental temperature rises;

FIG. 5 is a circuit diagram illustrating the configuration of a gas sensor 2 according to a second embodiment of the technology according to the present disclosure;

FIG. 6 is a flowchart for explaining the operation of the gas sensor 2;

FIG. 7 is a timing chart for explaining a first modification of the operation of the gas sensor 2;

FIG. 8 is a timing chart for explaining a second modification of the operation of the gas sensor 2; and

FIG. 9 is a timing chart for explaining a method of estimating the environmental temperature Temp3 in the third modification of the operation of the gas sensor 2.

### DETAILED DESCRIPTION OF THE EMBODIMENTS

Some embodiments of the present disclosure will be explained below in detail with reference to the accompanying drawings.

### First Embodiment

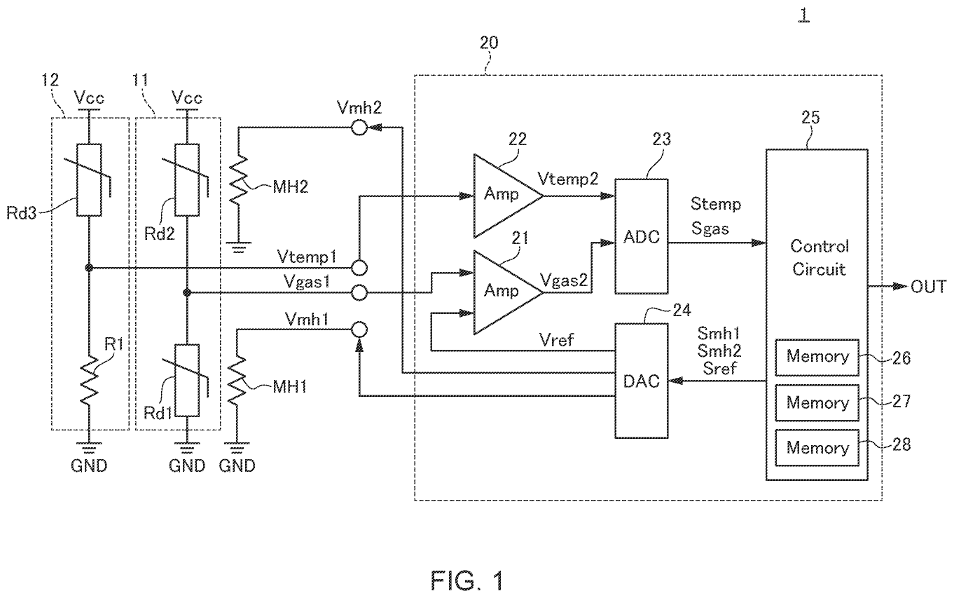

FIG. 1 is a circuit diagram illustrating the configuration of a gas sensor 1 according to a first embodiment of the technology according to the present disclosure.

As illustrated in FIG. 1, the gas sensor 1 according to the first embodiment has a sensor part 11, a temperature sensor part 12, heater resistors MH1 and MH2, and a signal processing circuit 20. Although not particularly limited, the gas sensor 1 according to the first embodiment is configured to detect the concentration of CO₂ gas in the atmosphere.

The sensor part 11 is constituted by a heat conduction type gas sensor for measuring the concentration of CO₂ gas as a gas to be measured and includes thermistors Rd1 and Rd2 which are connected in series. The temperature sensor part 12 includes a thermistor Rd3 and a fixed resistor R1 which are connected in series. The thermistors Rd1 to Rd3 are each a thermosensitive element made of a material having a negative resistance temperature coefficient, such as a composite metal oxide, amorphous silicon, polysilicon, or germanium. The thermistors Rd1 and Rd2 are both configured to detect the concentration of CO₂ gas but have mutually different operating temperatures as will be described later. The thermistor Rd3 functions as a temperature sensor for detecting environmental temperature. The environmental temperature refers to temperature in a measuring atmosphere. The thermistor Rd3 may be designed so as not to be influenced (or so as to be hardly influenced) by heating from the heater resistors MH1 and MH2, for example.

As illustrated in FIG. 1, the thermistors Rd1 and Rd2 are connected in series between a wire supplied with a power supply potential Vcc and a wire supplied with a ground potential GND. The thermistor Rd1 is heated to, for example, around 300° C. by the heater resistor MH1, and the thermistor Rd2 is heated to, for example, around 150° C. by the heater resistor MH2. When a gas concentration in the measuring atmosphere assumes a certain specific value, for example, when a CO₂ gas concentration in the measuring atmosphere assumes an average concentration value (about 400 ppm) in the atmosphere, the thermistor Rd1 is heated to, for example, 300° C. by the heater resistor MH1, and the thermistor Rd2 is heated to, for example, 150° C. by the heater resistor MH2. The thermistor Rd1 is designed to have a predetermined resistance value when heated to 300° C., and the thermistor Rd2 is designed to have a predetermined resistance value when heated to 150° C. A gas detection signal Vgas1 appears at the connection point between the thermistors Rd1 and Rd2.

When CO₂ gas is present in the measuring atmosphere in a state where the thermistor Rd2 as a thermosensitive element for detection is heated to around 150° C., heat dissipation characteristics of the thermistor Rd2 change according to the concentration of CO₂ gas. This change appears as a change in the temperature of the thermistor Rd2, i.e., the resistance value of the thermistor Rd2. Specifically, CO₂ gas is lower in heat dissipation than air, so that the temperature of the thermistor Rd2 increases as the concentration of CO₂ gas becomes high. Thus, for example, when the CO₂ gas concentration in the measuring atmosphere is higher than the average concentration in the atmosphere, the temperature of the thermistor Rd2 exceeds 150° C. As a result, the resistance value of the thermistor Rd2 lowers as compared with when the CO₂ gas concentration in the measuring atmosphere assumes the average concentration value in the atmosphere. On the other hand, when CO₂ gas is present in the measuring atmosphere in a state where the thermistor Rd1 as a thermosensitive element for reference is heated to around 300° C., heat dissipation characteristics of the thermistor Rd1 hardly change according to the concentration, and the temperature of the thermistor Rd1 also hardly changes. Accordingly, a change in the resistance value of the thermistor Rd1 heated to around 300° C. according to the CO₂ gas concentration is sufficiently smaller than a change in the resistance value of the thermistor Rd2 heated to around 150° C. according to the CO₂ gas concentration and may be imperceptible. The gas detection signal Vgas1 appearing at the connection point between the thermistors Rd1 and Rd2 is supplied to the signal processing circuit 20.

The thermistor Rd3 and fixed resistor R1 are connected in series between a wire supplied with a power supply potential Vcc and a wire supplied with a ground potential GND. A temperature detection signal Vtemp1 appears at the connection point between the fixed resistor R1 and the thermistor Rd3. The temperature detection signal Vtemp1 is supplied to the signal processing circuit 20.

The signal processing circuit 20 has amplifiers 21 and 22, an AD converter (ADC) 23, a DA converter (DAC) 24, and a control circuit 25. The amplifier 21 compares the gas detection signal Vgas1 and a reference voltage Vref to generate an amplified gas detection signal Vgas2. The amplifier 21 is a differential amplifier, for example, and amplifies a difference between the gas detection signal Vgas1 and the reference voltage Vref to generate and output the gas detection signal Vgas2. The amplifier 22 amplifies the temperature detection signal Vtemp1 to generate a temperature detection signal Vtemp2. The gas detection signal Vgas2 and temperature detection signal Vtemp2 are input to the AD converter 23. The AD converter 23 AD-converts the gas detection signal Vgas2 and temperature detection signal Vtemp2 to generate gas detection data Sgas and temperature detection data Stemp as digital values. The digital data Sgas and Stemp are supplied to the control circuit 25.

The control circuit 25 generates heater indicating values Smh1, Smh2, and a reference value Sref as digital values and supplies these values to the DA converter 24. The DA converter 24 coverts the heater indicating values Smh1, Smh2, and reference value Sref to heater voltages Vmh1, Vmh2, and reference voltage Vref, respectively. The heater voltages Vmh1 and Vmh2 are applied to the heater resistors MH1 and MH2, respectively, to heat the thermistors Rd1 and Rd2. The reference voltage Vref is supplied to the amplifier 21.

The control circuit 25 has memories 26 to 28. The memory 26 stores therein a calculation expression or conversion table indicating the relation between the gas detection data Sgas and an output signal OUT. The output signal OUT is a voltage value or a digital value indicating the concentration of CO₂ gas as a gas to be measured and is output outside the gas sensor 1.

The memory 27 stores therein a calculation expression or conversion table indicating the relation between the environmental temperature indicated by the temperature detection data Stemp and heater indicating values Smh1 and Smh2. The reason why such a calculation expression or conversion table is required is that the heater indicating values Smh1 and Smh2 required to heat the thermistors Rd1 and Rd2 to their desired temperature ranges (for example, around 300° C. and around 150° C., respectively) change with the environmental temperature.

The memory 28 stores therein a calculation expression or conversion table indicating the relation between the environmental temperature indicated by the temperature detection data Stemp and reference value Sref. The reason why such a calculation expression or conversion table is required is that the center level (for example, ½ Vcc level) of the gas detection signal Vgas1 changes with the environmental temperature. The calculation expression or conversion table to be stored in the memory 28 is generated based on the result of actual measurement performed before shipping. For example, the gas detection signal Vgas1 is measured at multiple environmental temperatures while keeping the CO₂ gas concentration constant, and based on the obtained measurement results, the calculation expression or conversion table can be generated.

The following describes the operation of the gas sensor 1 according to the first embodiment.

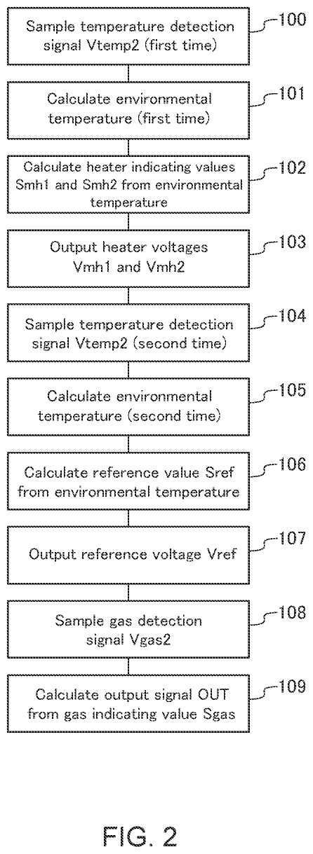

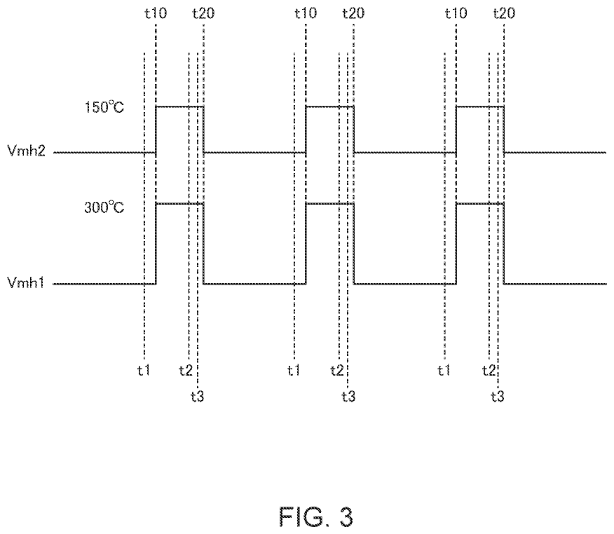

FIG. 2 is a flowchart for explaining the operation of the gas sensor 1. FIG. 3 is a timing chart for explaining the operation of the gas sensor 1.

The signal processing circuit 20 included in the gas sensor 1 performs a first sampling process of the temperature detection signal Vtemp2 (step 100). Specifically, the first sampling of the temperature detection signal Vtemp2 is performed as follows: the temperature detection signal Vtemp1 is converted to the temperature detection signal Vtemp2 using the amplifier 22, then the obtained temperature detection signal Vtemp2 is further converted to the temperature detection data Stemp using the AD converter 23, and the obtained temperature detection data Stemp is taken into the control circuit 25. This first sampling of the temperature detection signal Vtemp2 is performed at the timing t1 illustrated in FIG. 3. The timing t1 is a timing immediately before the timing t10 at which the heater resistors MH1 and MH2 start heating the thermistors Rd1 and Rd2, respectively.

Then, the control circuit 25 calculates the environmental temperature based on the temperature detection data Stemp (step 101) and then refers to the memory 27 to thereby calculate the heater indicating values Smh1 and Smh2 from the ambient temperature (step 102). The heater indicating values Smh1 and Smh2 are converted, by the DA converter 24, to the heater voltages Vmh1 and Vmh2, respectively, which are then applied to the heater resistors MH1 and MH2 (step 103), respectively, whereby the heating of the thermistors Rd1 and Rd2 is started. The thermistors Rd1 and Rd2 start being heated at the timing t10 illustrated in FIG. 3.

The thermistors Rd1 and Rd2 do not become stable in temperature from the heating start timing t10 until the passage of a predetermined time, thus requiring a predetermined standby time from the heating start until the start of sampling of the gas detection signal Vgas2.

Then, the signal processing circuit 20 performs a second sampling process of the temperature detection signal Vtemp2 (step 104). The second sampling of the temperature detection signal Vtemp2 is performed at the timing t2 illustrated in FIG. 3. Then, the control circuit 25 calculates the environmental temperature again based on the temperature detection data Stemp obtained by the second sampling (step S105) and then refers to the memory 28 to thereby calculate the reference value Sref from the environmental temperature (step 106). The reference value Sref is converted, by the DA converter 24, to the reference voltage Vref, which is then applied to the amplifier 21 (step 107), whereby the amplifier 21 becomes able to compare the gas detection signal Vgas1 with the reference voltage Vref and outputs the gas detection signal Vgas2 as a result of the comparison.

Then, the signal processing circuit 20 performs sampling of the gas detection signal Vgas2 (step 108). Specifically, the sampling of the gas detection signal Vgas2 is performed as follows: the gas detection signal Vgas2 output from the amplifier 21 as a result of comparison between the gas detection signal Vgas1 and reference voltage Vref is converted to the gas detection data Sgas using the AD converter 23, and the obtained gas detection data Sgas is taken into the control circuit 25. This sampling of the gas detection signal Vgas2 is performed at the timing t3 illustrated in FIG. 3. The timing t3 is a timing advanced, by at least the above-mentioned standby time, from the timing t10 at which the heating of the thermistors Rd1 and Rd2 is started.

The control circuit 25 refers to the memory 26 to calculate the output signal OUT from the gas detection data Sgas (step 109) and outputs the calculated output signal OUT to the outside. After the timing t3, the control circuit 25 resets the heater indicating values Smh1 and Smh2 and stops heating the thermistors Rd1 and Rd2. The heating of the thermistors Rd1 and Rd2 is stopped at the timing t20 illustrated in FIG. 3.

By repeating the above operations at a predetermined period, the concentration of the gas to be detected contained in the environment can be detected in real time.

As described above, in the present embodiment, the sampling of the temperature detection signal Vtemp2 is performed twice at the timings t1 and t2. Then, heating temperature of the thermistors Rd1 and Rd2 is controlled based on the result of the sampling performed at the timing t1, and the level of the reference voltage Vref is controlled based on the result of the sampling performed at the timing t2. In addition, since the timing t2 is set immediately before the timing t3 at which the sampling of the gas detection signal Vgas2 is performed, influence of a change in the environmental temperature is reduced.

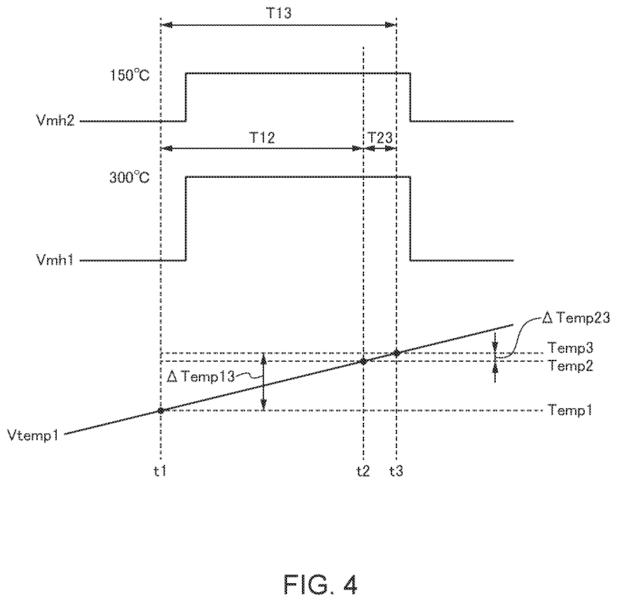

FIG. 4 is a timing chart for explaining influence of a temperature error when the environmental temperature rises.

In the example illustrated in FIG. 4, the environmental temperature at the timing t1 is Temp1, the environmental temperature at the timing t2 is Temp2 (>Temp1), and the environmental temperature at the timing t3 is Temp3 (>Temp2). In this case, if not only the temperature control of the thermistors Rd1 and Rd2 but also the level control of the reference voltage Vref is performed based on the result of the sampling performed at the timing t1, the error ΔTemp13 occurs, due to long standby time, in the environmental temperature at the timing t3 at which the sampling of the gas detection signal Vgas2 is actually performed. On the other hand, in the present embodiment, the level control of the reference voltage Vref is performed based on the result of the sampling performed at the timing t2 immediately before the timing t3, so that the temperature error ΔTemp23 is smaller than the temperature error ΔTemp13.

The time difference T23 between the timings t2 and t3 is preferably as small as possible within a range allowing the signal processing circuit 20 to complete its control operation and is more preferably smaller than ½ of the time difference T13 between the timings t1 and t3. In other words, the time difference T23 between the timings T2 and t3 is preferably smaller than the time difference T12 between the timings t1 and t2.

The concentration measurement method for the gas to be detected illustrated in FIGS. 2 and 3 can be applied to generation of the calculation expression or conversion table to be stored in the memory 28. This can enhance the accuracy of the calculation expression or conversion table to be stored in the memory 28.

### Second Embodiment

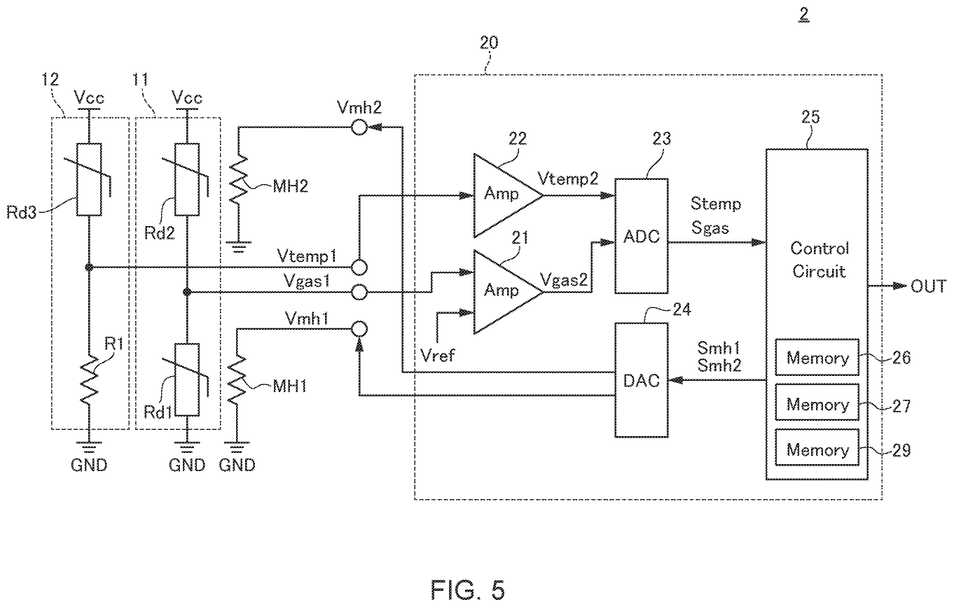

FIG. 5 is a circuit diagram illustrating the configuration of a gas sensor 2 according to a second embodiment of the technology according to the present disclosure.

As illustrated in FIG. 5, the gas sensor 2 according to the second embodiment differs from the gas sensor 1 according to the first embodiment in that a memory 29 is provided in place of the memory 28 and that the DA converter 24 does not output the reference voltage Vref. Other basic configurations are the same as those of the gas sensor 1 according to the first embodiment, so the same reference numerals are given to the same elements, and overlapping description will be omitted. The memory 29 stores therein a calculation expression or conversion table indicating the relation between the environmental temperature indicated by the temperature detection data Stemp and a correction value C to be described later.

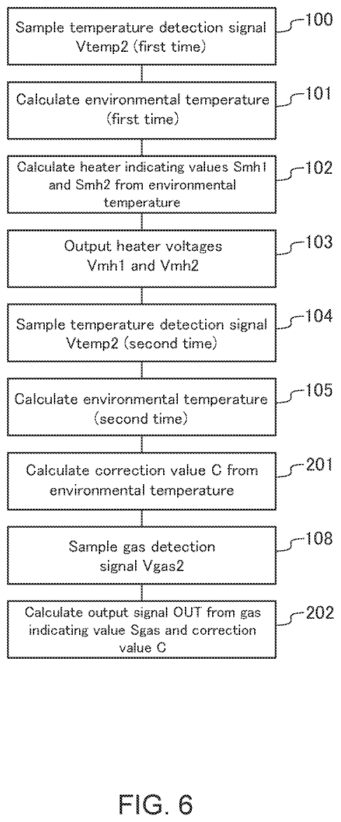

FIG. 6 is a flowchart for explaining the operation of the gas sensor 2. The operation timing is as illustrated in FIG. 3.

The operations from steps 100 to 105 are performed, and the environmental temperature is calculated based on the result of the second sampling. Then, the correction value C is calculated by referring to the memory 29 (step 201). The correction value C is a component corresponding to a deviation in the reference voltage Vref output to the amplifier 21 due to the environmental temperature. In the present embodiment, the level of the reference voltage Vref to be output to the amplifier 21 may be a fixed value irrelevant to the environmental temperature.

Then, the signal processing circuit 20 performs sampling of the gas detection signal Vgas2 to acquire the gas detection data Sgas (step 108) and then calculates the output signal OUT based on the gas detection data Sgas and correction value C (step S202). Specifically, the output signal OUT may be calculated as follows: the memory 26 is referred to, to calculate the output signal OUT from the gas detection data Sgas, and then the output signal OUT is corrected based on the correction value C; alternatively, the gas detection data Sgas may be corrected first based on the correction value C, followed by calculation of the output signal OUT from the corrected gas detection data Sgas with reference to the memory 26.

As described above, in the present embodiment, instead of controlling the level of the reference voltage Vref based on the result of the sampling performed at the timing t2, the correction value C is generated based on the result of the sampling performed at the timing t2, followed by calculation of the output signal OUT using the correction value C. This enhances freedom in timing for performing the second sampling.

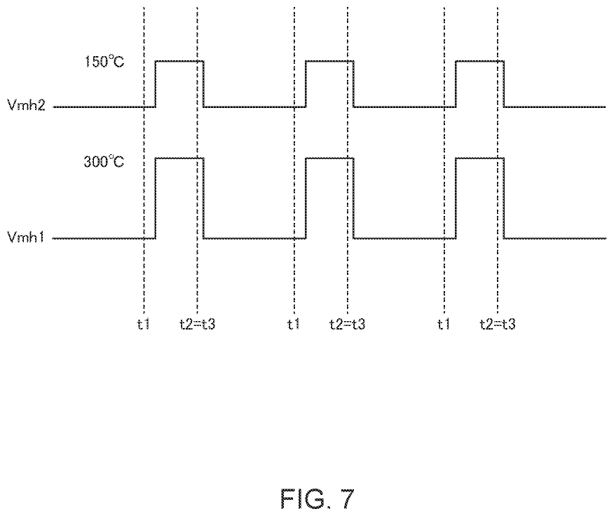

For example, as a first modification illustrated in FIG. 7, the timing t2 at which the second sampling of the temperature detection signal Vtemp2 is performed and the timing t3 at which the sampling of the gas detection signal Vgas2 is performed may be substantially the same. This makes the time difference T23 shown in FIG. 4 substantially zero, and thus the temperature error ΔTemp23 also becomes substantially zero.

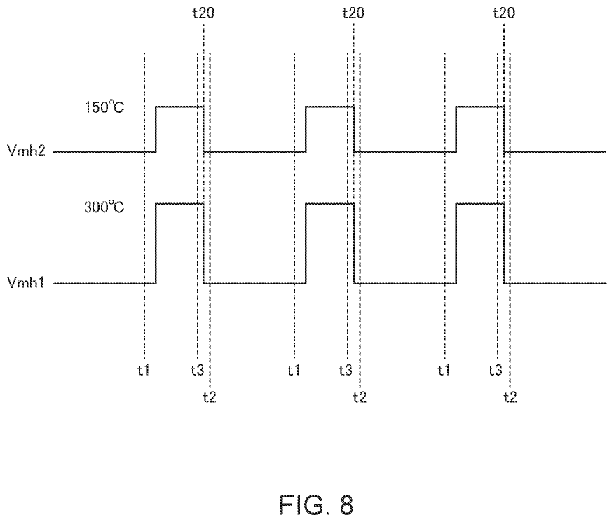

Alternatively, as a second modification illustrated in FIG. 8, the timing t2 at which the second sampling of the temperature detection signal Vtemp2 is performed may be later than the timing t3 at which the sampling of the gas detection signal Vgas2 is performed. In the example illustrated in FIG. 8, the timing t2 is later than the timing t20 at which the heating of the thermistors Rd1 and Rd2 is stopped. In this case, it is possible to reduce the detection error of the environmental temperature due to the heating of the thermistors Rd1 and Rd2.

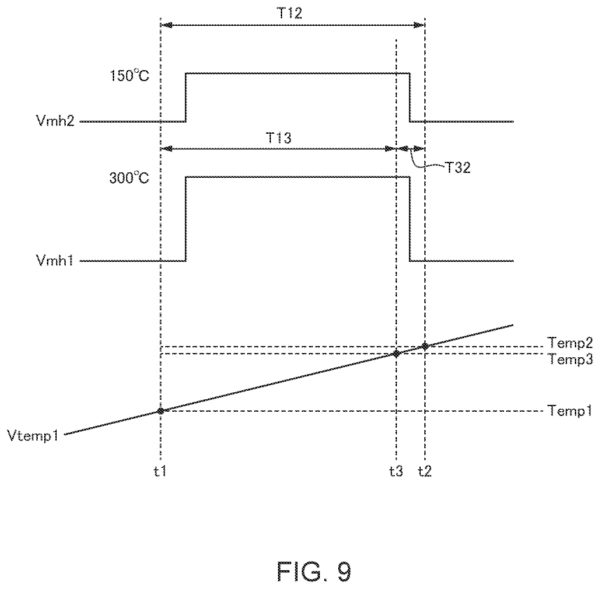

Further, as illustrated in FIG. 9, when the environmental temperature at the timing t1 is Temp1, and the environmental temperature at the timing t2 is Temp2 (>Temp1), an estimation value of the environmental temperature Temp3 at the timing t3 is calculated using the control circuit 25 based on the ratio between the time difference T12 between the timings t1 and t2, time difference T13 between the timings t1 and t3, and time difference T32 between the timings t3 and t2, whereby the correction value C can be calculated from the estimation value of the environmental temperature Temp3.

While the preferred embodiment of the present disclosure has been described, the present disclosure is not limited to the above embodiment, and various modifications may be made within the scope of the present disclosure, and all such modifications are included in the present disclosure.

For example, although the thermistor is used as a thermosensitive element in the above embodiment, the present invention is not limited to this.

The technology according to the present disclosure includes the following configuration examples but not limited thereto.

A gas sensor according to one aspect of the present disclosure includes: a gas sensor part that includes a thermosensitive element and generates a gas detection signal according to the concentration of a gas to be detected; a temperature sensor part that generates a temperature detection signal according to environmental temperature; a heater that heats the thermosensitive element; and a signal processing circuit that controls the heating temperature of the heater according to the temperature detection signal obtained at a first timing and calculates the concentration of the gas to be detected according to a temperature detection signal obtained at a second timing that is later than the first timing and a gas detection signal obtained at a third timing at which the thermosensitive element is heated by the heater. Thus, even with a change in the environmental temperature, a more accurate gas concentration can be calculated.

In the above gas sensor, the signal processing circuit may include a control circuit that calculates the level of a reference voltage according to the temperature detection signal obtained at the second timing and an amplifier that compares the gas detection signal obtained at the third timing and the reference voltage and calculate the concentration of the gas to be detected according to an output signal from the amplifier. This reduces signal processing burden on the control circuit.

In the above gas sensor, the signal processing circuit may calculate a correction value according to the temperature detection signal obtained at the second timing and calculate the concentration of the gas to be detected using the gas detection signal and the correction value. This enhances setting freedom of the second timing. In this case, the second timing and third timing may be the same, or the second timing may be later than the third timing.

What is claimed is:

1. A gas sensor comprising:
a gas sensor part including a thermosensitive element, the gas sensor part being configured to generate a gas detection signal according to a concentration of a gas to be detected;
a temperature sensor part configured to generate a temperature detection signal according to environmental temperature;
a heater configured to heat the thermosensitive element; and
a signal processing circuit configured to:
control a heating temperature of the heater according to the temperature detection signal obtained at a first timing; and
calculate the concentration of the gas to be detected according to the temperature detection signal obtained at a second timing that is later than the first timing and the gas detection signal obtained at a third timing at which the thermosensitive element is heated by the heater.

2. The gas sensor as claimed in claim 1, wherein the signal processing circuit includes:
a control circuit configured to calculate a level of a reference voltage according to the temperature detection signal obtained at the second timing; and
an amplifier configured to:
compare the gas detection signal obtained at the third timing and the reference voltage; and
calculate the concentration of the gas to be detected according to an output signal from the amplifier.

3. The gas sensor as claimed in claim 1, wherein the signal processing circuit is configured to:
calculate a correction value according to the temperature detection signal obtained at the second timing; and
calculate the concentration of the gas to be detected using the gas detection signal and the correction value.

4. The gas sensor as claimed in claim 3, wherein the second timing and third timing are a same.

5. The gas sensor as claimed in claim 3, wherein the second timing is later than the third timing.
